(19) United States
(12) Patent Application Publication
(10) Pub. No.: US 2006/0205010 A1
(43) Pub. Date: Sep. 14, 2006

(54) Methods of host cell protein analysis

(76) Inventors: Catherine Allioux, L'Isle-Adam (FR); Egisto Boschetti, Croissy sur Seine (FR); Virginie Brenac, Rueil Malmaison (FR); Patrick Santambien, Paris (FR); James Spencer, Frederick, MD (US)

Correspondence Address: QUINE INTELLECTUAL PROPERTY LAW GROUP, P.C., P O BOX 458, ALAMEDA, CA, 94501, US

(21) Appl. No.: 10/553,923

(22) PCT Filed: Apr. 19, 2004
(86) PCT No.: PCT/US04/12062

§ 371 Date: Oct. 21, 2005

Related U.S. Application Data
(60) Provisional application No. 60/464,902, filed on Apr. 22, 2003.

(51) Int. Cl. G01N 33/53

(52) U.S. Cl. 435/7.1

(57) ABSTRACT

The invention provides methods of determining the presence of host cell proteins in sample. The invention further provides methods of following the purification of target proteins. Kits and articles of manufacture are also provided.

### CROSS-REFERENCES TO RELATED APPLICATIONS

This application claims the benefit of U.S. Provisional Application No. 60/464,902, filed Apr. 22, 2003, the disclosure of which is incorporated by reference in its entirety for all purposes.

### COPYRIGHT NOTIFICATION

Pursuant to 37 C.F.R. § 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

The production of recombinant and other target proteins commonly includes expressing such proteins in host cells. Many of these proteins are intended for use as therapeutic agents in humans and as such must be highly purified. The manufacturing and purification process of these products leaves the potential for contamination by host cell proteins (or HCP) and other impurities from the host. Such contamination can result in adverse toxic or immunological reactions and thus it is desirable to reduce host cell contamination to the lowest levels possible.

To date, Western blotting and enzyme-linked immunosorbent assays (ELISA) are among the few methods that have been developed to detect and quantify traces of HCP. Both Western blotting and ELISA involve specific antibodies against targeted HCP. More specifically, Western blotting is a technique for detecting proteins in a mixture in which the proteins are separated electrophoretically and then transferred to a polymer sheet, which is exposed to a radiolabeled or enzyme-conjugated antibody specific for the protein of interest. Western blots have relatively limited sensitivity. To illustrate, for a typical mini-electrophoresis, the sensitivity of this technique is generally only to about 1 ng. In addition, Western blots are highly labor intensive to perform, characterized by subjective interpretation, and essentially qualitative in nature.

ELISA is an assay for detecting either antibodies or antigens by use of an enzyme-linked antibody and a substrate that forms a colored reaction product. Various enzymes have been used for ELISA, including alkaline phosphatase, horseradish peroxidase, and p-nitrophenyl phosphatase. While ELISA-based methods are more quantitative than Western blotting, they also have significant limitations. For example, the calorimetric or fluorescent signals produced by these assays yield only global results of the host cell proteins present in a sample. Further, the technique does not produce chromatographic discrimination among contaminants. This prevents, for example, the development of improved purification techniques that would more completely separate most representative HCP components, e.g., prior to performing enzymatic immunoassays. Moreover, the ELISA determination is only valid for each single HCP that has a corresponding antibody.

Western blotting and ELISA in the context of HCP detection are further referred to in, e.g., Eaton (1995) “Host cell contaminant protein assay development for recombinant biopharmaceuticals,” J. Chromatogr. 705:105-114, which is one of the first general attempts to measure HCP by an immunoassay, Dagouassat et al. (2001) “Development of a quantitative assay for residual host cell proteins in a recombinant subunit vaccine against human respiratory syncytial virus,” J. Immunol. Methods. 251:151-159, which describes a quantitative immunoligand assay for residual host cell proteins from a vaccine preparation with an alleged sensitivity of between 10 to 30 ppm, and Wan et al. (2002) “An enzyme-linked immunosorbent assay for host cell protein contaminants in recombinant PEGylated staphylokinase mutant SY161,” J. Pharm. Biomed. Anal. 28:953-963, which describes an immunoassay for the quantitation of HCP present in a recombinant PEGylated staphylokinase produced in a culture of E. coli with a purported sensitivity of between 1 to 100 ng/ml.

In view of the foregoing discussion, it is apparent that there is a substantial need for methods of determining the presence of host cell proteins in samples that, e.g., have improved sensitivities relative to preexisting techniques, provide rapid results, and are not merely qualitative. These and a variety of other features of the present invention will become apparent upon complete review of the following disclosure.

### SUMMARY OF THE INVENTION

The present invention generally relates to the science of bioprocessing. More specifically, the invention provides methods of detecting and quantitatively analyzing host cell proteins and other impurities in biological samples. The sensitivity of the methods described herein is typically in the range of femtomoles and thus are well suited for assaying samples from intended injectable biologicals, such as cell culture supernatants comprising recombinant proteins. The high throughput methods of the present invention typically include detecting host cell proteins that have been specifically captured on solid supports utilizing various detection methods, including mass spectrometry. In addition, the invention also provides articles of manufacture and kits for performing the methods described herein.

In one aspect, the present invention relates to a method for determining the presence of host cell proteins in a sample. The method includes (a) capturing host cell proteins from a sample onto a solid support with at least one affnity reagent that specifically binds host cell proteins. The sample is typically selected from, e.g., cell culture supernatant, organ extracts, a sample derived from a transgenic animal, a sample derived from a transgenic plant, a sample derived from a transgenic egg, a biological fluid, or the like. The method also includes (b) detecting the captured host cell proteins. In preferred embodiments, for example, the host cell proteins are detected by mass spectrometry.

The affinity reagents used in the methods of the invention and the approaches to capturing them on solid supports include various embodiments. Exemplary affinity reagents utilized in these methods are optionally selected from, e.g., monoclonal antibodies, polyclonal antibodies, phage display proteins, aptamers, affibodies, chemical ligands, peptides, and combinations thereof. Optionally, the affinity reagent includes IgG immunoglobulins. In some embodiments, the host cell proteins are captured on a solid support derivatized with the affinity reagent. In other embodiments, the host cell proteins are bound to the affinity reagent and the affinity reagent is subsequently captured on the solid support. In these embodiments, the solid support is optionally a chromatographic resin derivatized with a capture molecule that binds the affinity reagent. To further illustrate, the affinity reagent is optionally an antibody and the capture molecule is, e.g., Protein A, Protein G, a mercaptoheterocyclic ligand, or the like. In still other embodiments, the solid support is a surface enhanced laser desorption/ionization (or SELDI) biochip derivatized with a capture molecule that binds the affinity reagent. In these embodiments, the affinity reagent is similarly optionally an antibody and the capture molecule is optionally Protein A, Protein G, a mercaptoheterocyclic ligand, or the like. In another embodiment, the host cell protein affinity reagent may be coupled to a bridging element such as biotin or streptavidin, and the solid support could contain a moiety that binds to this element.

The solid supports used in these methods for determining the presence of host cell proteins in a sample also include various embodiments. For example, the solid support optionally includes a chromatographic resin (e.g., a material selected from ceramic, glass, metal, an organic polymer, and combinations thereof). In these embodiments, detecting generally includes washing unbound molecules from the resin, eluting the captured host cell proteins from the resin, and detecting the eluted host cell proteins. In some embodiments, the solid support includes a protein biochip. In these embodiments, the host cell proteins are typically detected by, e.g., a gas phase ion spectrometry method, an optical method, an electrochemical method, atomic force microscopy, a radio frequency method, etc. Optionally, the solid support includes a surface enhanced laser desorption/ionization biochip on which the affinity reagent is immobilized before or after capturing the host cell proteins. SELDI typically includes applying a matrix material to the biochip before laser desorption/ionization. As an additional option, the SELDI biochip includes a surface-enhanced neat desorption (SEND) surface.

The present invention also provides methods of following purification of a target protein. These methods include (a) profiling a sample that includes the target protein (e.g., a naturally occurring protein, or a recombinant or otherwise artificially evolved protein) at one step of a purification process in which profiling includes detecting the target protein in the sample and detecting host cell proteins in the sample using the methods for determining the presence of host cell proteins in a sample described above. The methods also include (b) subjecting the target protein to at least one purification step, and (c) profiling the sample that includes the target protein after the purification step in which profiling comprises detecting the target protein in the sample and detecting host cell proteins in the sample similarly using the method for determining the presence of host cell proteins in a sample described above. Additionally, the method includes (d) comparing the relative amounts of the target protein and the host cell proteins in the sample detected by profiling.

In other aspects, the invention provides articles of manufacture and kits. For example, an article of manufacture of the invention typically includes a solid support, at least one affinity reagent bound to the solid support in which the affinity reagent specifically binds host cell proteins and host cell proteins bound to the affinity reagent. The kits of the invention generally include (a) a solid support derivatized with a reactive moiety or a capture molecule that specifically binds at least one affinity reagent. These kits also generally include (b) instructions to capture host cell proteins from a sample with the affinity reagent, which affinity reagent specifically binds the host cell proteins, and to immobilize the captured host cell proteins on the solid support.

### BRIEF DESCRIPTION OF THE DRAWINGS

FIG. 1 schematically depicts one embodiment of a surface enhanced laser desorption/ionization assay to determine the presence of host cell proteins in a sample.

FIG. 2 schematically shows another embodiment of a surface enhanced laser desorption/ionization assay to determine the presence of host cell proteins in a sample.

FIG. 3 schematically illustrates a surface enhanced laser desorption/ionization time-of-flight mass spectrometry system.

FIG. 4 schematically depicts a representative example information appliance or digital device in which various aspects of the present invention may be embodied.

### DETAILED DISCUSSION OF THE INVENTION

I. Definitions

As used herein, the terms set forth with particularity below and grammatical variations used herein have the following definitions. If not otherwise defined, all terms used herein have the meaning commonly understood by a person skilled in the art to which this invention pertains.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOP mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm² to 50 mJ/mm². Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Solid support” refers to a substrate having a surface on which to bind (e.g., directly or via a capture molecule or other reactive moiety) or otherwise present an adsorbent, such as an affinity reagent. Exemplary solid supports include probes, microtiter plates and chromatographic resins.

“Chromatographic resin” or “resin” refers to solid supports that typically comprise insoluble materials (e.g., polymeric materials) or particles having surfaces on which affinity reagents (e.g., antibodies, affibodies, aptamers, etc.) can be immobilized directly or via a linker or capture molecule, such as Protein A, Protein G, a mercaptoheterocyclic ligand, etc. Suitable resin materials include, but are not limited to, glass, silica, controlled pore glass (CPG), polystyrene, polystyrene/latex, polyacrylate, polyacrylamide, agar, agarose, chemically modified agars and agaroses, carboxyl modified polytetraflouethylene, nylon and nitrocellulose. These solid supports can be biological, nonbiological, organic, inorganic, or a combination of any of these, existing as beads, particles, strands, precipitates, gels, spheres, etc., depending upon the particular application. For example, polymer beads (e.g., polystyrene, polypropylene, latex, nylon and many others), silica or silicon beads, clay or clay beads, ceramic beads, glass beads, magnetic beads, metallic beads, inorganic compound beads, and organic compound beads can be used. An enormous variety of these materials are commercially available, e.g., those typically used for chromatography, as well as those commonly used for affinity purification. Exemplary commercial suppliers include, e.g., Promega Corp., the Baxter Immunotherapy Group, Sigma-Aldrich, Inc., and others.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a protein-nucleic acid conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Adsorption” refers to detectable non-covalent binding of an analyte to an adsorbent or capture reagent.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. SEND is further described in U.S. Pat. No. 5,719,060 and U.S. patent application 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through brealing a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface and, therefore, function as probes.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, WCX-2, CM-10, IMAC-3, IMAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 biochip has quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu++ and Ni++, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application US 2003 0032043 A1 (Pohl and Papanu, “Latex Based Adsorbent Chip,” Jul. 16, 2002); U.S. patent application 60/350,110 (Um et al., “Hydrophobic Surface Chip,” Nov. 8, 2001); U.S. patent application 60/367,837, (Boschetti et al., “Biochips With Surfaces Coated With Polysaccharide-Based Hydrogels,” May 5, 2002) and U.S. patent application entitled “Photocrosslinked Hydrogel Surface Coatings” (Huang et al., filed Feb. 21, 2003).

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

The “sensitivity” of a device or method is a measure of its ability to discriminate between small differences in analyte concentrations.

“Biological sample” and “sample” identically refer to a sample derived from at least a portion of an organism capable of replication. As used herein, a biological sample can be derived from any of the known taxonomic kingdoms, including virus, prokaryote, single celled eukaryote and multicellular eukaryote. The biological sample can derive from the entirety of the organism or a portion thereof, including from a cultured portion thereof. Biological samples can be in any physical form appropriate to the context, including homogenate, subcellular fractionate, lysate and fluid.

“Biomolecule” refers to a molecule that can be found in, but need not necessarily have been derived from, a biological sample. This includes molecules, such as nucleotides, amino acids, sugars, fatty acids, steroids, nucleic acids, polypeptides, peptides, peptide fragments, carbohydrates, lipids, and combinations of these (e.g., glycoproteins, ribonucleoproteins, lipoproteins, or the like).

The terms “polypeptide”, “peptide”, and “protein” are used interchangeably herein to refer to a naturally-occurring or synthetic polymer comprising amino acid monomers (residues), where amino acid monomer here includes naturally-occurring amino acids, naturally-occurring amino acid structural variants, and synthetic non-naturally occurring analogs that are capable of participating in peptide bonds. Polypeptides can be modified, e.g., by the addition of carbohydrate residues to form glycoproteins. The terms “polypeptide,” “peptide” and “protein” include glycoproteins as well as non-glycoproteins.

A “host cell protein” refers to any protein derived from a host cell system that one desires to detect. Host cell proteins typically include, but are not necessarily limited to, those proteins, which are native to the host cell. For example, biopharmaceutical products, such as recombinant or other target proteins expressed in a host cell system are typically separated from host cell proteins and other impurities through various purification processes during product manufacture. Generally the intent is to isolate a target protein from host cell proteins in a purification process.

A “target protein” refers to any protein (or set of proteins) of interest that is sought to be retained in a given process, such as a purification process. The target protein may be a recombinant protein, an artificially evolved protein or a native protein of interest. To illustrate, a target protein expressed in a host cell system is typically separated from host cell proteins and other impurities, and retained for subsequent use, e.g., as a therapeutic compound. Methods to artificially evolve proteins and nucleic acids that encode artificially evolved proteins are generally known in the art. Target proteins can also be naturally occurring (i.e., not artificially evolved) in a given host organism.

“Polynucleotide” and “nucleic acid” equivalently refer to a naturally-occurring or synthetic polymer comprising nucleotide monomers (bases). Polynucleotides include naturally-occurring nucleic acids, such as deoxyribonucleic acid (“DNA”) and ribonucleic acid (“RNA”), as well as nucleic acid analogs. Nucleic acid analogs include those which include non-naturally occurring bases, and those in which nucleotide monomers are linked other than by the naturally-occurring phosphodiester bond. Nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like.

“Receptor” refers to a molecule, typically a macromolecule, that can be found in, but need not necessarily have been derived from, a biological sample, and that can participate in specific binding with a ligand. The term further includes fragments and derivatives that remain capable of specific ligand binding.

“Ligand” refers to any compound that can participate in specific binding with a designated receptor or antibody.

“Antibody” refers to a polypeptide substantially encoded by at least one immunoglobulin gene or fragments of at least one immunoglobulin gene, that can participate in specific binding with a ligand. The term includes naturally-occurring forms, as well as fragments and derivatives. Fragments within the scope of the term as used herein include those produced by digestion with various peptidases, such as Fab, Fab′ and F(ab)′2 fragments, those produced by chemical dissociation, by chemical cleavage, and recombinantly, so long as the fragment remains capable of specific binding to a target molecule, such as a host cell protein. Typical recombinant fragments, as are produced, e.g., by phage display, include single chain Fab and scFv (“single chain variable region”) fragments. Derivatives within the scope of the term include antibodies (or fragments thereof) that have been modified in sequence, but remain capable of specific binding to a target molecule, including interspecies chimeric and humanized antibodies. As used herein, antibodies can be produced by any known technique, including harvest from cell culture of native B lymphocytes, hybridomas, recombinant expression systems, by phage display, or the like.

“Antigen” refers to a ligand that can be bound by an antibody. An antigen need not be immunogenic. The portions of the antigen that make contact with the antibody are denominated “epitopes”.

“Specific binding” refers to the ability of at least two molecular species simultaneously present in a heterogeneous (inhomogeneous) sample to bind to one another preferentially over binding to other molecular species in the sample. For example, an antibody specifically binds to one or more antigens (e.g., host cell proteins, etc.) bearing the epitope for which the antibody has antigenic specificity. Typically, a specific binding interaction will discriminate over adventitious binding interactions in the reaction by at least two-fold, more typically more than 10- to 100-fold. When used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

The term “attached,” as used herein, encompasses interactions including, but not limited to, covalent bonding, ionic bonding, chemisorption, physisorption, and combinations thereof.

II. Introduction

The present invention relates to the qualitative and quantitative determination of host cell proteins (HCP) and other process-related impurities in samples derived from diverse biological materials, including cell culture media. More specifically, the multiplexed detection methods of the invention combine the specificity of immunochemical reactions in the capture of HCP with the resolving power and sensitivity of various approaches to analyte detection, including mass spectrometry. Typically, the biological sample under consideration includes a target protein (e.g., a recombinant protein, etc.) that is being purified from host cell proteins and other contaminants present in the particular biological sample. Host cell proteins are proteins that can be released as a consequence of, e.g., natural secretion during cell life, cell lysis subsequent to death or cell lysis during downstream cell processing such as separation or induced physically or chemically lysis to release produced target protein. HCP and other impurities are sought to be removed during purification processes in order to get the target protein in a pure state. However, in spite of complex purification schemes, traces of host cell proteins may still be present in the sample or non-covalently bound to the target protein. As HCP are antigens that are typically incompatible as components of injectable biopharmaceutical formulations, it is desirable to identify and quantify them at various stages of a purification process.

In overview, the methods of the invention include a method for determining the presence of host cell proteins in samples and a method of following purification of a target protein. The method of determining the presence of HCP includes (a) capturing host cell proteins from a sample onto a solid support with at least one affinity reagent that specifically binds HCP. The method additionally includes (b) detecting the captured host cell proteins, e.g., by mass spectrometry or another approach to analyte detection. The method of following purification of a target protein includes (a) profiling a sample comprising the target protein at one step of a purification process in which the profiling comprises detecting the target protein in the sample and detecting host cell proteins in the sample using the method for determining the presence of HCP in samples referred to above. This method also includes (b) subjecting the target protein to a purification step, and (c) profiling the sample comprising the target protein after the purification step. Again, the profiling includes detecting the target protein in the sample and detecting HCP in the sample using the method for determining the presence of host cell proteins in samples described above. In addition, the method of following target protein purification also includes (d) comparing the relative amounts of the target protein and the host cell proteins in the sample detected by profiling. Each of these aspects, including exemplary variations, of the methods of the present invention are described in greater detail below. Additional details related to the present invention are also provided in, e.g., U.S. application Ser. No. 10/124,626, entitled “Methods for monitoring polypeptide production and purification using surface enhanced laser desorption/ionization mass spectrometry,” filed Apr. 16, 2002 by Boschetti et al.

The methods of the invention provide many advantages over various preexisting analytical techniques. To illustrate, compared to classical electrophoresis (e.g., mono- or bi-dimensional), the methods described herein are significantly more sensitive, are capable providing quantitative information, and can be utilized to make comparisons with patterns in a database. In addition, the methods of the present invention yield quantitative information and are not affected by high levels of protein expression, unlike Western blotting methods. To further illustrate, compared to ELISA analysis, the present determination typically provides information on the number and the amount of each single host cell protein present in a sample.

III. Sources and Preparation of Biological Samples

Host cell proteins and other non-target chemical species can be qualitatively and/or quantitatively detected in essentially any sample using the methods of the present invention. In preferred embodiments, the methods of the invention are utilized at selected stages of a given bioprocessing procedure (e.g., the production and purification of a therapeutic polypeptide or other biopharmaceutical, or of an agriculturally-, industrially-, or otherwise commercially-relevant compound) to inform on the purity level of, e.g., the batch at the particular stage in the process being assayed.

Target proteins, such as therapeutic proteins are typically naturally occurring in a given host organism, or the ultimate products of forced molecular evolutionary processes (e.g., nucleic recombination, mutagenesis, or other techniques known in the art) and expressed in, e.g., a heterologous host organism (e.g., eukaryotic cells and organisms, such as transgenic plants or animals, Chinese hamster ovary (CHO) cells, and fungal cells (e.g., Pichia pastoris, etc.), or prokaryotic cells (e.g., bacterial cells, such as Escherichia coli, etc.). They also exist in any cell-free expression system. Further, the samples utilized in performing the methods described herein are optionally derived from cell culture supernatant, e.g., when host organisms secrete target polypeptides into the surrounding culture medium. If a host organism does not secrete the target polypeptide into the surrounding medium, then samples are typically derived from lysates of the host organism. Cell lysates can be produced by shearing, centrifugation, and/or other cell harvesting techniques, which are generally known in the art.

General texts describing additional molecular biological techniques useful herein, including host organism selection, cell culture, and sample collection include Berger and Kimmel, Guide to Molecular Cloning Techniques, Methods in Enzymology, Vol. 152, Academic Press, Inc. (Berger), Sambrook et al., Molecular Cloning—A Laboratory Manual (2nd Ed.), Vol. 1-3, Cold Spring Harbor Laboratory (1989) (Sambrook), and Current Protocols in Molecular Biology, F. M. Ausubel et al. (Eds.), Current Protocols, a joint venture between Greene Publishing Associates, Inc. and John Wiley & Sons, Inc. (supplemented through 2000) (Ausubel). Methods of transducing cells, including plant and animal cells, with nucleic acids are generally available, as are methods of expressing proteins encoded by such nucleic acids. In addition to Berger, Ausubel and Sambrook, useful general references for culturing animal cells include Freshney, Culture of Animal Cells, a Manual of Basic Technique, 3rd Ed., Wiley-Liss (1994)(Freshney), Humason, Animal Tissue Techniques, 4^{th} Ed., W.H. Freeman and Company (1979), and Ricciardelli et al., In Vitro Cell Dev. Biol. 25:1016-1024 (1989). References for plant cell cloning, culture and regeneration include Payne et al., Plant Cell and Tissue Culture in Liquid Systems, John Wiley & Sons, Inc. (1992)(Payne) and Gamborg and Phillips (Eds.), Plant Cell, Tissue and Organ Culture, Fundamental Methods, Lab Manuals Series, Springer-Verlag (1995)(Gamborg).

Mass cell culture techniques are widely known in the science of bioprocessing. In particular, additional details relating to cell culture (including culturing cells of bacterial, plant, animal (especially mammalian) and archebacterial origin), culture media, and culture equipment are provided in, e.g., Fiechter (Ed.), Advances in Biochemical Engineering—Biotechnology: Bioprocess Design and Control, Springer-Verlag, Inc. (1993), Kargi, Bioprocess Engineering, 2^{nd}, Prentice Hall (2001), Buckland (Ed.), Cell Culture Engineering, Kluwer Academic Publishers (1995), Doran, Bioprocess Engineering Principles, Academic Press, Inc. (1995), Vieth, Bioprocess Engineering: Kinetics, Mass Transport, Reactors, and Gene Expression, John Wiley & Sons, Inc. (1994), Butler, Animal Cell Culture and Technology: The Basics, Oxford University Press, Inc. (1998), and Davis (Ed.), Basic Cell Culture: A Practical Approach, 2^{nd}, Oxford University Press, Inc. (2001).

The samples used in the methods of this invention are also optionally derived from other biological material sources. This includes biological fluids such as blood, serum, saliva, urine, prostatic fluid, seminal fluid, seminal plasma, lymph, lung/bronchial washes, mucus, feces, nipple secretions, sputum, tears, egg whites or yolks (e.g., from naturally occurring or transgenic eggs) or the like. It also includes extracts from biological samples, such as organ extracts, etc. In addition, biological samples such as these are optionally collected according to any known technique, such as venipuncture, biopsy, or the like. The specific exemplary sample sources listed herein are offered to illustrate but not to limit the present invention. Additional sources of samples are known in the art and are readily obtainable.

Target proteins are optionally recovered and purified from cell cultures or other sample sources by any of a number of methods well known in the art, including electrophoresis, chromatography, precipitation, dialysis, filtration, centrifugation, crystallization and/or precipitation. More specifically, purification techniques, such as ultra-centrifugation, ammonium sulfate or ethanol precipitation, acid extraction, ion exchange chromatography, high performance liquid chromatography, size exclusion chromatography, phosphocellulose chromatography, hydrophobic interaction chromatography (e.g., as with C₁-C₁₈ resins), affinity chromatography (e.g., as with immunoaffinity, immobilized metals, dyes, or other tagging systems), hydroxylapatite chromatography, and/or lectin chromatography are optionally used. Preferably, the sample is in a liquid form from which solid materials (e.g., cellular debris, etc.) have been removed. In addition to the references noted herein, a variety of purification methods are well known in the art, including, e.g., those set forth in Sandana, Bioseparation of Proteins, Academic Press, Inc. (1997), Bollag et al., Protein Methods, 2^{nd} Ed., Wiley-Liss (1996), Walker, The Protein Protocols Handbook, Humana Press (1996), Harris and Angal, Protein Purification Applications: A Practical Approach, IRL Press (1990), Harris and Angal (Eds.), Protein Purification Methods: A Practical Approach, IRL Press (1989), Scopes, Protein Purification: Principles and Practice, 3^{rd} Ed., Springer Verlag (1993), Janson and Ryden, Protein Purification: Principles, High Resolution Methods and Applications, 2nd Ed., Wiley-VCH (1998), Walker, Protein Protocols on CD-ROM, Humana Press (1998), Satinder Ahuja ed., Handbook of Bioseparations, Academic Press (2000), and the references cited therein. For example, any of these exemplary purification techniques are optionally utilized in performing the method of following purification of a target protein described herein. One may desire to concentrate a sample when the target protein is in low abundance.

While a sample comprising the target protein can be analyzed directly, in certain embodiments, the methods include fractionating biomolecules in an initial sample by one or a combination of fractionation techniques described above or otherwise known in the art to be useful for separating biomolecules to collect a sample fraction that includes the target protein prior to mass profiling. Fractionation is typically utilized to decrease the complexity of analytes in the sample to assist detection and characterization of target proteins or impurities, such as host cell proteins. Moreover, fractionation protocols can provide additional information regarding physical and chemical characteristics of biomolecular components in a sample. For example, if a sample is fractionated using an anion-exchange spin column, and if a target protein is eluted at a certain pH, this elution characteristic provides information regarding binding properties of the target protein. In another example, a sample can be fractionated to remove proteins or other molecules in the sample that are present in a high quantity and/or which would otherwise interfere with the detection of a particular target protein or a trace impurity (e.g., host cell proteins that are expressed only at low levels in a particular host organism).

Prior to profiling biomolecule masses in a sample by gas phase ion spectroscopy, for example, proteins in the samples of the invention are optionally fragmented or digested. This approach is particularly useful when components (e.g., protein impurities, such as host cell proteins, etc.) of, e.g., a cell culture medium are to be identified. Fragmentation is optionally effected using any technique that produces peptide fragments from proteins in a sample. Many of these techniques are generally known in the art. For example, proteins are optionally fragmented enzymatically, chemically, or physically. In certain embodiments of the invention, target proteins and/or peptide fragments resulting from fragmentation are optionally modified to improve resolution upon detection. In other embodiments, the fragmentation of biomolecular components of a sample can be performed “on chip” in a SELDI environment by placing an aliquot of the sample on an adsorbent spot and adding, e.g., the proteolytic agent to the material on the spot. Additional details relating to the identification of biomolecules via fragmentation are described in, e.g., International Publication No. WO 02/074927 entitled “High accuracy protein identification” by Pham.

IV. Affinity Reagents

Essentially any affinity reagent or adsorbent is optionally adapted for use in the methods of the present invention. In preferred embodiments, host cell proteins are detected in samples using biospecfic adsorbents, which specifically bind these protein impurities. Exemplary biospecific adsorbents that are optionally utilized include, e.g., polypeptides, peptides, enzymes, receptors, monoclonal antibodies, polyclonal antibodies, phage display proteins, aptamers, affibodies, IgG immunoglobulins, chemical ligands, and combinations thereof. Additional details relating to affinity reagents are described herein, including in the definitions provided above.

In one preferred embodiment, for example, antibodies against all host cell antigens of the particular host organism are utilized as biospecific adsorbents. To illustrate, if a target protein is expressed in, e.g., CHO cells, Escherichia coli, or Pichia pastoris, then antibodies against the antigens of that particular cell type are utilized. Similarly, if a sample is derived from other biological sources, such as organ extracts or biological fluids, then antibodies against all antigens of the particular biological source are utilized. Exemplary biological sources, which are used to perform the methods described herein, are described further above. As mentioned above, antibodies used to practice the present invention can be monoclonal or polyclonal antibodies. Techniques are generally known and readily available in the art for raising antibodies which are highly specific for a particular species or other biological source. See, e.g., Harlow et al., Monoclonal Antibodies: A Laboratorv Manual, Cold Springs Harbor Laboratory Press (1988), Paul (Ed.), Fundamental Immunology, Lippincott Williams & Wilkins (1998), and Harlow et al., Using Antibodies: A Laboratory Manual, Cold Spring Harbor Laboratory Press (1998).

As referred to above, other exemplary biospecific adsorbents optionally include, e.g., aptamers, selected affibodies, chemical ligands, and selected peptides. Phage display is also optionally used for the preparation of antibody-like proteins. Certain of these are described further in, e.g., Nord et al. (1995) “A combinatorial library of an alpha-helical bacterial receptor domain,” Protein Eng 8:601-608, Nord et al. (1997) “Binding proteins selected from combinatorial libraries of an alpha-helical bacterial receptor domain,” Nature Biotechnol 15:772-777, Nygren et al. (1997) “Scaffolds for engineering novel binding sites in proteins,” Curr Opin Struct Biol 7:463-469, Gunneriusson et al. (1999) “Affinity maturation of a Taq DNA polymerase specific affibody by helix shuffling,” Protein Eng 12:873-878, Nord et al. (2001) “Recombinant human factor VIII-specific affinity ligands selected from phage-displayed combinatorial libraries of protein A,” Eur J Biochem 268:42694277, Eklund et al. (2002) “Anti-idiotypic protein domains selected from protein A-based affibody libraries,” Proteins 48:454-462, Karlstrom et al. (2001) “Dual-labeling of a binding protein allows for specific fluorescence detection of native protein,” Analytical Biochemistr=295:22-30, Samuelson et al. (2002) “Display or proteins on bacteria,” J. Biotechnol. 96:129-154, Kurtz et al. (2003) “Inhibition of an activated Ras protein with genetically selected peptide aptamers,” Biotechnol Bioeng 82(1):38-46, and Vairamani et al. (2003) “G-quadruplex formation of thrombin-binding aptamer detected by electrospray ionization mass spectrometry,” J Am Chem Soc 125(1):42-3.

V. Capture of Host Cell Proteins from Samples onto Solid Supports and Preparation for Analyte Detection

The affinity reagents of the invention are optionally bound to or otherwise immobilized on solid supports (e.g., probes, chromatographic resins, etc.) either before or after host cell proteins are captured with those affinity reagents. In certain embodiments, for example, the analysis of host cell proteins present in a given biological sample according to the present invention includes immobilizing (e.g., covalently, etc.) a mixture of affinity reagents, such as antibodies against the considered host cell proteins on the surface of a solid support, e.g., a reactive surface (e.g., a biochip such as a ProteinChip® Array or reactive beads or resin generally used, e.g., for affinity chromatography). The antibody mixture (e.g., anti-HCP antibodies) is, e.g., a mixture of polyclonal IgG produced by animal immunization (e.g., rabbit, goat, or the like). The immobilized IgG anti-HCP is typically contacted with the biological sample to be analyzed (e.g., samples from very crude cell culture supernatant, from purification fractions, from final purified materials, etc.) to effect capture of the host cell proteins present in the sample prior to analyte detection. In other embodiments, the mixture of polyclonal IgG and/or other affinity reagents is contacted with the biological sample to effect capture of the host cell proteins present in the sample prior to immobilizing the affinity reagent mixture on the particular solid support.

To further illustrate, if the solid support surface is on a probe, such as a ProteinChip® Array, for example, the probe surface is optionally directly analyzed by, e.g., SELDI TOF-MS. As described herein, this process generally includes loading energy adsorbing molecules (EAM) on the probe surface, followed by a drying operation, and analyzing the protein mixture by laser desorption/ionization mass spectrometry. If the solid support surface is on chromatography beads, for example, captured host cell proteins are typically desorbed and collected from the beads by an acidic or other treatment. The collected host cell protein fraction is then typically analyzed using various detection methods, including mass spectrometry (e.g., SELDI, MALDI, electrospray, etc.). Analyte detection is described further below.

Whether immobilized before or after capture, biological samples and affinity reagents are contacted or incubated together for a selected period of time (e.g., minutes, hours, days, etc.) in order to allow host cell proteins present in the sample to be captured or bound by the anti-HCP antibodies or other affinity reagents. Typically, samples and affinity reagents are contacted for a period of between about 30 seconds and about 12 hours, and preferably, between about 30 seconds and about 15 minutes. Furthermore, samples are generally contacted with affinity reagents under ambient temperature and pressure conditions. For some samples, however, modified temperature (typically between about 0° C. and about 100° C. and more typically 4° C. through 37° C.) and pressure conditions can be desirable, which conditions are determinable by those skilled in the art. Generally, a sample volume of about 1 μl to 500 μl is contacted with, e.g., an affinity reagent in a particular capture step. For example, the sample volume typically contains from a few attomoles to 100 picomoles of biomolecules (e.g., host cell proteins or other impurities). In embodiments in which samples and affinity reagents are immobilized after the capture step, affinity reagents are also typically provided in volumes of about 1 μl to 500 μl.

Essentially any method of immobilizing or attaching affinity reagents to solid supports (e.g., to derivatize the solid supports with the affinity reagents) is optionally utilized. For example, affinity reagents are optionally directly immobilized on a solid support surface or via a linker or capture molecule, such as Protein A, Protein G, a mercaptoheterocyclic ligand, or the like. Methods of immobilizing affinity reagents to solid supports are generally known in the art and are described further in, e.g., U.S. Patent Application 2003/0017464 (Pohl).

Probes suitable for use in the invention are described further herein, e.g., in the definitions provided above.

In certain embodiments, samples are analyzed without being fractionated prior to examination by an analytical technique, such as MALDI or SELDI. For example, a sample of a cell culture supernatant is optionally analyzed directly from a cell culture medium to assess the presence of host cell proteins or a secreted target protein. Another option includes, analyzing samples taken from various stages of a purification process in the absence of fractionation prior to, e.g., mass profiling or another method of detecting analytes.

Samples are optionally analyzed, according to the methods of the present invention, after fractionation of the samples (e.g., before or after being captured by affinity reagents, etc.). Fractionation of a sample aliquot typically increases the total information content about biomolecules present in the particular sample. For example, fractionation may result in the detection of trace impurities (e.g., host cell proteins) that would otherwise be undetectable, or not accurately detected, in an unfractionated sample by eliminating signals attributable to more abundant biomolecules that would otherwise suppress the signals of less abundant components. Further, biomolecules remaining in the sample after fractionation are typically detected with improved, e.g., mass accuracy as a result of an increased signal:noise ratio. The use of information about sample components from fractionated samples as well as unfractionated samples generally leads to a higher confidence level that a given target protein or impurity has been accurately detected.

The fractionation steps that generate sample fractions can be performed by, e.g., any of the purification/fractionation methods described above. For example, prior to spectrometrically profiling biomolecule masses in a particular sample, biomolecules in the sample are optionally separated into fractions using, e.g., centrifugation, dialysis, HPLC, SEC or the like. Typically, fractionated samples are then analyzed by various methods of detection, including traditional MALDI and other mass spectrometry approaches. Additional details relating to analyte detection are provided below.

In some embodiments, fractionating and analyzing the sample is performed by SELDI/retentate chromatography. Retentate chromatography involves directly contacting a sample with an adsorbent bound to a surface of a probe in which the adsorbent captures one or more biomolecular components, such as host cell proteins. This embodiment also includes removing non-captured material from the probe, e.g., by one or more washes prior to gas phase ion spectrometric analysis. Optionally, the sample is indirectly contacted with a probe surface after being contacted with, e.g., an affinity reagent bound to a chromatographic resin, which affinity reagent captures one or more components of the sample. In this embodiment, non-captured materials are optionally removed (e.g., by one or more washes) before or after the adsorbent is contacted with the probe surface. Additional details relating to retentate chromatography are provided in, e.g., U.S. Patent Application 20020177242 (Hutchens).

Washing to remove non-captured materials can be accomplished by, e.g., bathing, soaking, dipping, rinsing, spraying, or washing the surface of the solid support (e.g., probe, chromatographic resin, etc.) following exposure to the sample with an eluant. A microfluidics process is preferably used when an eluant is introduced to small spots (e.g., surface features) of affinity reagents on the probe. Typically, the eluant can be at a temperature of between 0° C. and 100° C., preferably between 4° C. and 37° C. Any suitable eluant (e.g., organic or aqueous) can be used to wash the substrate surface. For example, each of the one or more washes optionally includes an identical or a different elution condition relative to a preceding wash. Elution conditions typically differ according to, e.g., pH, buffering capacity, ionic strength, a water structure characteristic, detergent type, detergent strength, hydrophobicity, dielectric constant, concentration of at least one solute, or the like. Preferably, an aqueous solution is used. Exemplary aqueous solutions include a HEPES buffer, a Tris buffer, or a phosphate buffered saline, etc. To increase the wash stringency of the buffers, additives can be incorporated into the buffers. These include, but are not limited to, ionic interaction modifiers (both ionic strength and pH), water structure modifiers, hydrophobic interaction modifiers, chaotropic reagents, affinity interaction displacers. Specific examples of these additives can be found in, e.g., PCT publication WO 98/59360 (Hutchens and Yip). The selection of a particular eluant or eluant additives is dependent on other experimental conditions (e.g., types of adsorbents used or host cell proteins to be detected), and can be determined by those of skill in the art.

An option to detect molecules with very large masses, such as IgM antibodies, is to treat them in reducing conditions so as to produce smaller fragments. This results not from digestion, but rather from a dissociation of disulfide bonds (when present). In the case of antibody molecules, this produces heavy and light chains that are both smaller than the whole antibody and more easily detected by, e.g., mass spectrometry.

In certain embodiments of the invention, captured host cell proteins are desorbed and ionized from probe surfaces before being detected. Prior to desorption and ionization of biomolecules from a probe surface according to any of the methods described herein, energy absorbing molecules or matrix material is typically applied to a given sample on the substrate surface, usually after allowing the sample to dry. The energy absorbing molecules can assist absorption of energy from an energy source from a gas phase ion spectrometer, and can assist desorption of biomolecules from the probe surface. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in the art. See, e.g., U.S. Pat. No. 5,719,060 (Hutchens & Yip) for additional description of energy absorbing molecules.

The energy absorbing molecule and biomolecules in a given sample can be contacted in any suitable manner. For example, an energy absorbing molecule is optionally mixed with a sample and the mixture is placed on the probe surface, as in a traditional MALDI process. In another example, an energy absorbing molecule can be placed on the probe surface prior to contacting the probe with a sample. As an additional option, a fraction can be placed on the probe surface prior to contacting the probe with an energy absorbing molecule. Then, the biomolecule components in the sample can be desorbed, ionized and detected as described in detail below.

Optionally, multiple fractions of a given sample are analyzed in parallel. Additional options include analyzing unfractionated and fractionated samples in parallel. However, in other embodiments of the invention, these analyses can be performed in series. For example, a unfractionated sample aliquot can be placed on a spot and allowed to equilibrate. Then, the remaining liquid in the sample can be transferred to an adsorbent spot for fractionation by retentate chromatography.

VI. Analyte Detection

In preferred embodiments of the invention, biomolecules, such as host cell proteins and target proteins (e.g., recombinant proteins, etc.) in a sample are detected using gas phase ion spectrometry, and more preferably, using mass spectrometry. In one embodiment, matrix-assisted laser desorption/ionization (MALDI) mass spectrometry is used, e.g., to profile biomolecule masses in a sample. In MALDI, the sample is typically quasi-purified to obtain a fraction that essentially consists of the protein analyte to be detected using, e.g., protein separation methods such as dialysis, centrifugation, two-dimensional gel electrophoresis, HPLC, or the like. Biomolecule fractionation techniques are described in greater detail above. Additional details relating to MALDI and other variations of mass spectrometry techniques and instrumentation are included in, e.g., Skoog et al., Principles of Instrumental Analysis, 5^{th} Ed., Harcourt Brace & Co. (1998), Siuzdak, Mass Spectrometry for Biotechnology, Academic Press (1996), de Hoffmann et al., Mass Spectrometry: Principles and Applications, 2^{nd}, John Wiley & Sons, Inc. (2001), and Chapman, Mass Spectrometry of Proteins and Peptides, Vol. 146, Methods in Molecular Biology Series, Humana Press (2000).

The technique of MALDI is well known in the art. See, e.g., U.S. Pat. No. 5,045,694 (Beavis et al.), U.S. Pat. No. 5,202,561 (Gleissmann et al.), and U.S. Pat. No. 6,111,251 (Hillenkamp).

In certain preferred embodiments, SELDI mass spectrometry is optionally used to desorb and ionize biomolecules from probe surfaces. SELDI mass spectrometry is typically more sensitive than MA/DI mass spectrometry. Different versions of SELDI can be utilized to perform the methods of the invention. In general, SELDI includes the use of a probe comprising adsorbents (e.g., affinity reagents) to capture protein analytes, which are then optionally directly desorbed and ionized from the substrate surface during mass spectrometry. As described above, affinity reagents are optionally immobilized on probe surfaces before or after capturing host cell proteins. Since the probe surface in surface enhanced laser desorption/ionization captures sample components, a sample need not be quasi-purified as in MALDI. However, depending on the complexity of a sample and the type of adsorbents used, it is typically desirable to prepare a sample aliquot with reduced complexity by, e.g., removing non-captured materials prior to surface enhanced laser desorption/ionization analysis.

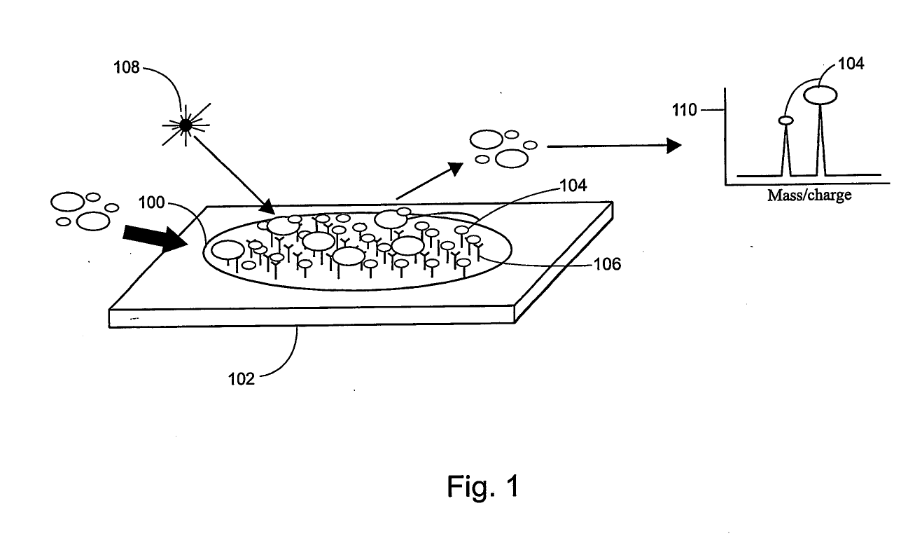

To further illustrate aspects of SELDI, FIG. 1 schematically shows an assay of an unfractionated sample that includes affinity reagent 106 (e.g., an antibody specific for a host cell protein) on biochip 102. Affinity reagents are described further above. As additionally described above, biomolecules 104 (e.g., host cell proteins) in the sample are not washed after being placed on affinity reagent 106 which is bound to surface feature 100 of biochip 102. Incident photon energy from laser 108 causes the desorption and ionization of biomolecules 104, which are then detected in a mass spectrometer to produce mass spectrum 110.

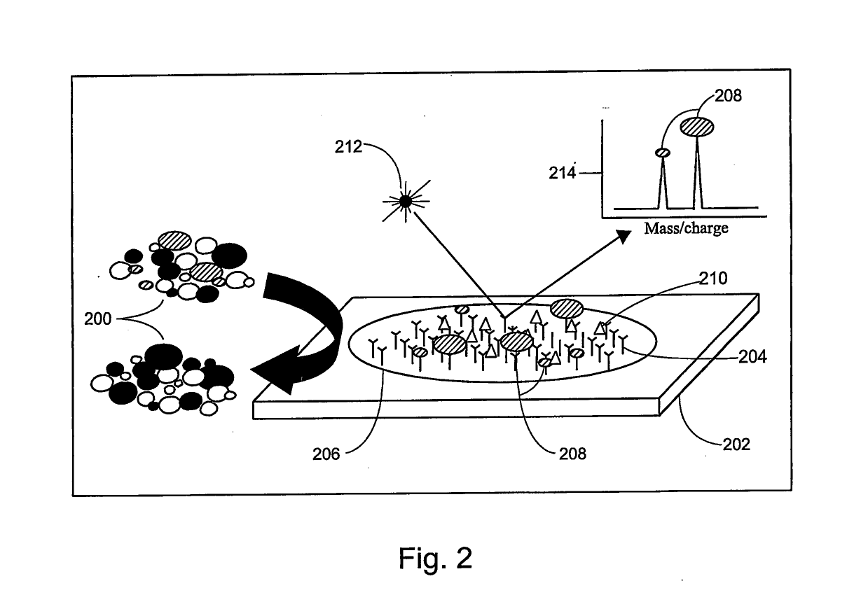

FIG. 2 schematically illustrates a surface enhanced laser desorption/ionization assay of a sample, such as one taken from a cell culture supernatant that includes a mixture of secreted target proteins and host cell proteins. As depicted, sample 200 is applied to biochip 202 which includes affinity reagent 204 (e.g., an antibody specific for a host cell protein) bound to surface feature 206. Components of sample 200 that are not bound to affinity reagent 204 are washed away (e.g., eluted or the like) from biochip 202 prior to mass analysis, as described above. Following capture and washing of host cell proteins 208 in sample 200, energy absorbing molecules 210 (not shown in FIG. 1) are added to biochip 202 to absorb energy from ionization source 212 (i.e., a laser) to aid desorption of host cell proteins 208 from the surface of biochip 202. Mass spectrum 214 is produced by mass spectral analysis of desorbed/ionized host cell proteins 208.

Optionally, any suitable gas phase ion spectrometer is used as long as it allows biomolecular components on the substrate to be resolved and detected. For example, in certain embodiments the gas phase ion spectrometer is a mass spectrometer. In a typical mass spectrometer, a probe comprising biomolecules on its surface is introduced into an inlet system of the mass spectrometer. The biomolecules are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomolecules or other substances will typically involve detection of signal intensity. This, in turn, can reflect the quantity and character of biomolecules bound to the substrate. Any of the components of a mass spectrometer (e.g., a desorption source, a mass analyzer, a detector, etc.) can be combined with other suitable components described herein or others known in the art in embodiments of the invention.

In a preferred aspect, a laser desorption time-of-flight (TOF) mass spectrometer is used in certain embodiments of the invention. In laser desorption mass spectrometry, a substrate or a probe comprising biomolecular components (e.g., bound host cell proteins) is introduced into an inlet system. The materials are desorbed and ionized into the gas phase by incident laser energy from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of proteins or protein fragments of specific mass-to-charge ratios.

In another preferred aspect, a tandem mass spectrometer is used in various embodiments of the invention. Tandem mass spectrometers can usefully be selected from the group that includes orthogonal quadrupole time-of-flight (Qq-TOF), ion trap (IT), ion trap time-of-flight (IT-TOF), time-of-flight time-of-flight (TOF-TOF), and ion cyclotron resonance (ICR) varieties. Presently preferred is an orthogonal Qq-TOF MS. Tandem mass spectrometers and associated instrumentation which can be adapted to perform the methods described herein are described further in, e.g., Patent Application Publication No. US 2002/0182649 by Weinberger et al., which published Dec. 5, 2002.

In another embodiment, an ion mobility spectrometer or total ion current measuring device is optionally used to detect biomolecular components.

As alternatives to gas phase ion spectrometry detection methods (described above), any detection method or device compatible with the assay system and the nature of the biomolecule of interest is optionally used in practicing the present invention. To illustrate, upon capture on a biochip, analytes can be detected by a variety of detection methods including, e.g., optical methods, electrochemical methods, atomic force microscopy, radio frequency methods, etc. Optical methods include, for example, detection of fluorescence, luminescence, chemiluminescence, absorbance (e.g., ultraviolet or visible light), reflectance, transmittance, birefringence, refractive index or diffraction (e.g., surface plasmon resonance, ellipsometry, resonant mirror methods, grating-coupled waveguide methods, interferometry, multi-polar resonance spectroscopy, etc.). Optical methods include microscopy (both confocal and non-confocal), imaging methods and non-imaging methods. Immunoassays in various formats (e.g., ELISA) are also optionally used for detection of analytes captured on a solid phase. Suitable immunoassays that can be adapted for use in practicing the present invention are described further in, e.g., Wild (Ed.), The Immunoassay Handbook, 2^{nd}, Nature Publishing Group (2000), Law (Ed.), Immunoassay: A Practical Guide, Taylor & Francis, Inc. (1996), and Johnstone et al., Immunochemistry in Practice, 3^{rd}, Blackwell Publishers (1996). Exemplary electrochemical methods include voltametry and amperometry methods, while exemplary radio frequency methods include multipolar resonance spectroscopy. Additional details relating to gas phase ion spectrometry and other methods of detection are described in, e.g., Skoog et al., Principles of Instrumental Analysis, 5^{th} Ed., Harcourt Brace College Publishers (1998) and Currell, Analytical Instrumentation: Performance Characteristics and Quality, John Wiley & Sons, Inc. (2000).

The detectors utilized in practicing the invention typically further comprise interfaced digital computers, e.g., to control device operation (e.g., ion generation in a gas phase ion spectrometer, etc.) and to participate in data acquisition and analysis. Analysis software can be local to the computer or can be remote, but communicably accessible to the computer. For example, the computer can have a connection to the internet permitting use of analytical packages such as Protein Prospector, PROWL, or the Mascot Search Engine, which are available on the world wide web. The analysis software can also be remotely resident on a LAN or WAN server. Exemplary systems that include digital computers are described further below.

VII. Data Analysis

In overview, when the presence of host cell proteins is detected, for example, using mass spectrometry (e.g., either on the surface of a probe, such as a ProteinChip® Array, or desorbed from chromatographic beads or resin), the results would show a pattern of different peaks of given molecular masses. Detected masses typically correspond to single host cell proteins (except, e.g., for multiple charged-species, subunits or fragments of the same protein, etc.). The size of the signal compared to a standard curve is generally proportional to the amount of the particular host cell protein. Pattern analysis software containing data related to host cell proteins typically informs on the identity of detected host cell proteins, provides quantitative information regarding these contaminating proteins, and informs on the effectiveness of the purification steps for the particular target protein. Furthermore, it also informs qualitatively as well quantitatively about the best conditions of culture or of extraction to reduce the presence of HCP contamination in a target protein production process.

Data generated by desorption and detection of biomolecules, such as host cell proteins is optionally analyzed using any suitable method, e.g., to identify and/or quantify detected components. In one embodiment, data is analyzed with the use of a logic device, such as a programmable digital computer that is included, e.g., as part of a system. Systems are described further below. The computer generally includes a computer readable medium that stores logic instructions of the system software. Certain logic instructions are typically devoted to memory that includes the location of each feature on a probe, the identity of the adsorbent at that feature, the elution conditions used to wash the adsorbent, or the like. The computer also typically includes logic instructions that receives as input, data on the strength of the signal at various molecular masses received from a particular addressable location or surface feature on the probe, and instructions for entering data into a database. This data generally indicates the number and masses of components detected, including the strength of the signal generated by each component.

To further illustrate, data generation in mass spectrometry typically begins with the detection of ions by an ion detector. A typical laser desorption mass spectrometer can employ a nitrogen laser at 337.1 nm. A useful pulse width is about 4 nanoseconds. Generally, power output of about 1-25 μJ is used. Ions that strike the detector generate an electric potential that is digitized by a high speed time-array recording device that digitally captures the analog signal. Ciphergen's ProteinChip® system employs an analog-to-digital converter (ADC) to accomplish this. The ADC integrates detector output at regularly spaced time intervals into time-dependent bins. The time intervals typically are one to four nanoseconds long. Furthermore, the time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation, baseline subtraction, high frequency noise filtering.

TOP-to-M/Z transformation involves the application of an algorithm that transforms times-of-flight into mass-to-charge ratio (M/Z). In this step, the signals are converted from the time domain to the mass domain. That is, each time-of-flight is converted into mass-to-charge ratio, or M/Z. Calibration can be done internally or externally. In internal calibration, the sample analyzed contains one or more analytes of known M/Z. Signal peaks at times-of-flight representing these massed analytes are assigned the known M/Z. Based on these assigned M/Z ratios, parameters are calculated for a mathematical function that converts times-of-flight to M/Z. In external calibration, a function that converts times-of-flight to M/Z, such as one created by prior internal calibration, is applied to a time-of-flight spectrum without the use of internal calibrants.

Baseline subtraction improves data quantification by eliminating artificial, reproducible instrument offsets that perturb the spectrum. It involves calculating a spectrum baseline using an algorithm that incorporates parameters such as peak width, and then subtracting the baseline from the mass spectrum.

High frequency noise signals are eliminated by the application of a smoothing function. A typical smoothing function applies a moving average function to each time-dependent bin. In an improved version, the moving average filter is a variable width digital filter in which the bandwidth of the filter varies as a function of, e.g., peak bandwidth, generally becoming broader with increased time-of-flight. See, e.g., WO 00/70648, Nov. 23, 2000 (Gavin et al., “Variable Width Digital Filter for Time-of-flight Mass Spectrometry”).

A computer can transform the resulting spectrum into various formats for displaying. In one format, referred to as “spectrum view or retentate map,” a standard spectral view can be displayed, wherein the view depicts the quantity of analyte reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes which are up- or down-regulated between samples.

Analysis generally involves the identification of peaks in the spectrum that represent signal from an analyte. Peak selection can, of course, be done by eye. However, software is available as part of Ciphergen's ProteinChip® software that can automate the detection of peaks. In general, this software functions by identifying signals having a signal-to-noise ratio above a selected threshold and labeling the mass of the peak at the centroid of the peak signal. In one useful application many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes.

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, which is herein incorporated by reference in its entirety.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are in U.S. Provisional Patent Application Nos. 60/249,835, filed on Nov. 16, 2000, and 60/254,746, filed on Dec. 11, 2000, and U.S. Non-Provisional patent application Ser. No. 09/999,081, filed Nov. 15, 2001, and Ser. No. 10/084,587, filed on Feb. 25, 2002. All of these U.S. Provisional and Non Provisional Patent Applications are herein incorporated by reference in their entirety for all purposes.

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

VIII. Systems

The present invention also relates to systems capable of providing mass spectral profiles of host cell proteins and other components in a sample according to the methods described herein. Systems typically include one or more affinity reagents (e.g., affinity reagents bound to a probe surface, affinity reagents bound to chromatographic beads, or the like) capable of capturing host cell proteins in the sample under various conditions and a gas phase ion spectrometer (e.g., a mass spectrometer, such as a laser desorption/ionization mass spectrometer) able to profile masses of captured biomolecules to provide mass data. Systems also typically include one or more processors (e.g., in a computer or other logic device) operably connected to the gas phase ion spectrometer. Processors are optionally internal or external to the gas phase ion spectrometer. System software typically includes logic instructions, e.g., capable of quantifying detected host cell proteins, capable of determining closeness-of-fit between one or more detected biomolecule masses in sets of mass data and database entries, or the like.

Various software packages are currently available for querying databases, improving the speed of mass spectrometric protein identification processes, and otherwise integrating mass spectrometry into bioinformatics. For example, Mascot is a search engine that uses mass spectrometry data to identify proteins from primary sequence databases. See, e.g., Perkins et al. (1999) “Probability-based protein identification by searching sequence databases using mass spectrometry data,” Electrophoresis 20(18):3551-3567. Another exemplary software package that is useful for performing the methods of the present invention includes ProFound, which performs rapid database searching combined with Bayesian statistics for protein identification. Profound is described further in, e.g., Zhang and Chait (2000) “ProFound-An expert system for protein identification using mass spectrometric peptide mapping information,” Anal. Chem. 72:2482-8249, Zhang and Chait (1998) “ProFound-An expert system for protein identification,” Proceedings of the 46th ASMS Conference on Mass Spectrometry and Allied Topics, Orlando, Fla., p. 969, and Zhang and Chait (1995) “Protein identification by database searching: a Bayesian algorithm,” Proceedings of the 43rd ASMS Conference on Mass Spectrometry and Allied Topics, Atlanta, Georgia, p. 643.

The invention also provides for the storage and retrieval of a collection of data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor).

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like in which the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected binding functionality) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

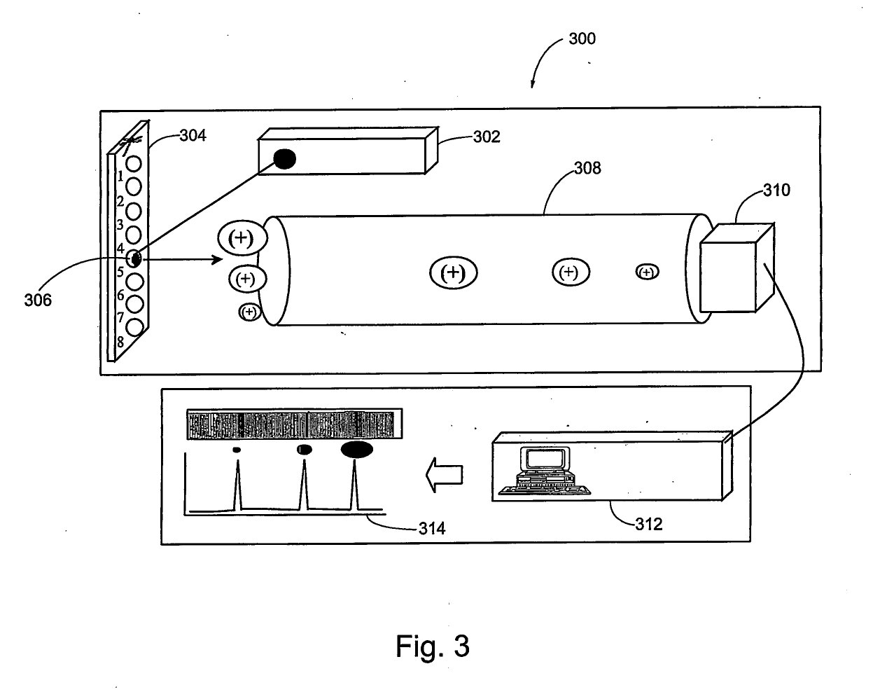

FIG. 3 schematically illustrates an exemplary surface enhanced laser desorption/ionization time-of-flight mass spectrometry system 300. As shown, photon energy produced by laser source 302 impacts biochip 304 at surface feature 306, which includes a selected affinity reagent with captured biomolecules (e.g., host cell proteins). The photon energy causes captured biomolecules at surface feature 306 to desorb and ionize. The desorbed ions are then accelerated through flight tube/mass analyzer 308. Ions are separated according to mass/charge ratios, which as depicted is simply the mass of the ionic species, because each ion is singly charged. As further illustrated, smaller ions travel faster than larger ions, thereby resolving the species according to mass. Ions produce a detectable signal at detector 310 which signal is processed by information appliance or digital device 312 to generate mass spectrum 314.

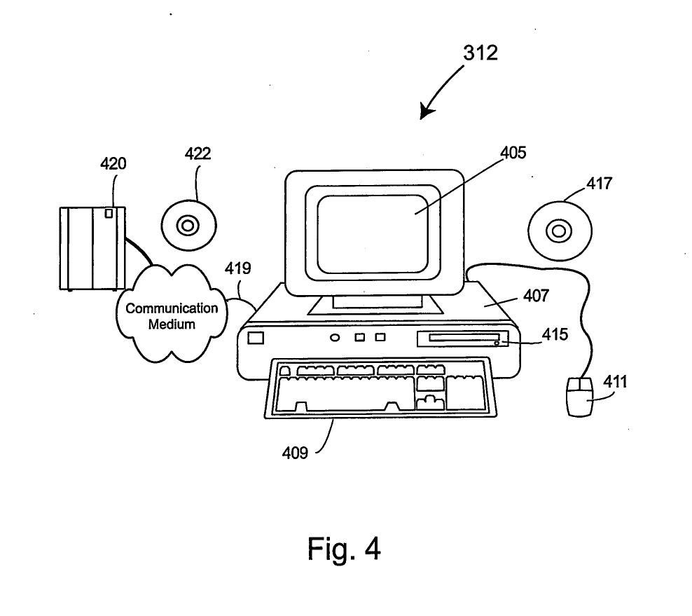

FIG. 4 is a schematic showing additional representative details of information appliance 312 from FIG. 3 in which various aspects of the present invention may be embodied. As will be understood by practitioners in the art from the teachings provided herein, the invention is optionally implemented in hardware and/or software. In some embodiments, different aspects of the invention are implemented in either client-side logic or server-side logic. As will be understood in the art, the invention or components thereof may be embodied in a media program component (e.g., a fixed media component) containing logic instructions and/or data that, when loaded into an appropriately configured computing device, cause that device to perform according to the invention. As will also be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

FIG. 4 shows information appliance or digital device 312 that may be understood as a logical apparatus that can read instructions from media 417 and/or network port 419, which can optionally be connected to server 420 having fixed media 422. Apparatus 312 can thereafter use those instructions to direct server or client logic, as understood in the art, to embody aspects of the invention. One type of logical apparatus that may embody the invention is a computer system as illustrated in 312, containing CPU 407, optional input devices 409 and 411, disk drives 415 and optional monitor 405. Fixed media 417, or fixed media 422 over port 419, may be used to program such a system and may represent a disk-type optical or magnetic media, magnetic tape, solid state dynamic or static memory, or the like. In specific embodiments, the invention may be embodied in whole or in part as software recorded on this fixed media Communication port 419 may also be used to initially receive instructions that are used to program such a system and may represent any type of communication connection. Optionally, the invention is embodied in whole or in part within the circuitry of an application specific integrated circuit (ACIS) or a programmable logic device (PLD). In such a case, the invention may be embodied in a computer understandable descriptor language, which may be used to create an ASIC, or PLD.

IX. Kits

The present invention also provides kits for determining the presence of host cell proteins in a sample and for following the purification of target proteins. Kits generally include one or more solid supports derivatized with a reactive moiety or a capture molecule that specifically binds at least one affinity reagent. For example, the solid support is optionally a probe (e.g., a biochip) as described herein. In some embodiments, solid supports are chromatographic resins or beads. The kits of the invention optionally also include affinity reagents, such as antibodies to host cell proteins, e.g., either bound to the capture molecule on the solid support or packaged separately. Suitable affinity reagents are described in greater detail above. Kits may further include a pre-fractionation spin column (e.g., K-30 size exclusion column).

In addition, kits also generally include instructions (e.g., in the form of a label or a separate insert) to capture host cell proteins from a sample with the affinity reagent, and to immobilize the captured host cell proteins on the solid support. The instructions may also include other operational parameters. For example, the kit may have standard instructions informing a consumer how to wash the probe after, e.g., a sample is contacted on the probe. In another example, the kit may have instructions for pre-fractionating a sample to reduce complexity of proteins in the sample. Optionally, the kit may further include a standard or control information for comparison with information derived from test samples.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method for determining the presence of host cell proteins in a sample, the method comprising:
(a) capturing host cell proteins from a sample onto a solid support with at least one affinity reagent that specifically binds host cell proteins; and,
(b) detecting the captured host cell proteins.

2. The method of claim 1, wherein the sample is selected from cell culture supernatant, organ extracts, a sample derived from a transgenic animal, a sample derived from a transgenic plant, a sample derived from a transgenic egg or a biological fluid.

3. The method of claim 1, wherein the affinity reagent is selected from monoclonal antibodies, polyclonal antibodies, phage display proteins, aptamers, affibodies, chemical ligands, peptides, and combinations thereof.

4. The method of claim 1, wherein the affinity reagent comprises IgG immunoglobulins.

5. The method of claim 1, wherein the solid support comprises a protein biochip.

6. The method of claim 5, wherein the host cell proteins are detected by a gas phase ion spectrometry method, an optical method, an electrochemical method, atomic force microscopy, or a radio frequency method.

7. The method of claim 1, wherein the solid support comprises a surface enhanced laser desorption/ionization biochip on which the affinity reagent is immobilized before or after capturing the host cell proteins.

8. The method of claim 7, wherein surface enhanced laser desorption/ionization comprises applying a matrix material to the biochip before laser desorption/ionization.

9. The method of claim 7, wherein the surface enhanced laser desorption/ionization biochip comprises a surface-enhanced neat desorption surface.

10. The method of claim 1, wherein the solid support comprises a chromatographic resin.

11. The method of claim 10, wherein the resin comprises a material selected from ceramic, glass, metal, an organic polymer, and combinations thereof.

12. The method of claim 10, wherein detecting comprises washing unbound molecules from the resin, eluting the captured host cell proteins from the resin, and detecting the eluted host cell proteins.

13. The method of claim 10, wherein the solid support is a chromatographic resin derivatized with a capture molecule that binds the affinity reagent.

14. The method of claim 13, wherein the affinity reagent is an antibody and the capture molecule is Protein A, Protein G, or a mercaptoheterocyclic ligand.

15. The method of claim 1, wherein the host cell proteins are captured on a solid support derivatized with the affinity reagent.

16. The method of claim 1, wherein the host cell proteins are bound to the affinity reagent and the affinity reagent is subsequently captured on the solid support.

17. The method of claim 16, wherein the solid support is a surface enhanced laser desorption/ionization biochip derivatized with a capture molecule that binds the affinity reagent.

18. The method of claim 17, wherein the affinity reagent is an antibody and the capture molecule is Protein A, Protein 0, or a mercaptoheterocyclic ligand.

19. The method of claim 1, wherein the host cell proteins are detected by mass spectrometry.

20. A method of following purification of a target protein comprising:
(a) profiling a sample comprising the target protein at one step of a purification process, wherein profiling comprises detecting the target protein in the sample and detecting host cell proteins in the sample using the method according to claim 1;
(b) subjecting the target protein to at least one purification step;
(c) profiling the sample comprising the target protein after the purification step, wherein profiling comprises detecting the target protein in the sample and detecting host cell proteins in the sample using the method according to claim 1; and,
(d) comparing the relative amounts of the target protein and the host cell proteins in the sample detected by profiling.

21. The method of claim 20, wherein the target protein comprises a recombinant protein.

22. An article of manufacture comprising a solid support, at least one affinity reagent bound to the solid support, wherein the affinity reagent specifically binds host cell proteins and host cell proteins bound to the affinity reagent.

23. A kit comprising:
(a) a solid support derivatized with a reactive moiety or a capture molecule that specifically binds at least one affinity reagent; and,
(b) instructions to capture host cell proteins from a sample with the affinity reagent, which affinity reagent specifically binds the host cell proteins, and to immobilize the captured host cell proteins on the solid support.
